(19) United States
(12) Patent Application Publication
(10) Pub. No.: US 2006/0099697 A1
(43) Pub. Date: May 11, 2006

(54) Anaerobic fermenter

(76) Inventor: Reinhart Von Nordenskjold, Egmating-Munster (DE)

Correspondence Address: SMITH-HILL AND BEDELL, P.C., 16100 NW CORNELL ROAD, SUITE 220, BEAVERTON, OR, 97006, US

(21) Appl. No.: 10/534,889

(22) PCT Filed: Nov. 7, 2003
(86) PCT No.: PCT/EP03/12473

§ 371 Date: May 12, 2005

(30) Foreign Application Priority Data
Nov. 13, 2002 (EP) 02025529.5

(51) Int. Cl. C12P 5/00

(52) U.S. Cl. 435/166

(57) ABSTRACT

The invention relates to a device for anaerobically fermenting substances comprising a preacidifier inside of which the substances are subjected to a preacidification, a fermenter inside of which the preacidified substances ferment, and transfer means for transferring the substances out of the preacidifier and into the fermenter. The device is characterized in that the transfer means are designed for selectively transferring sufficiently preacidified substances. The invention also relates to a method for anaerobically fermenting substances involving a preacidification during which the substances are preacidified by means of a preacidifier, a fermentation during which the preacidified substances ferment inside a fermenter, and a transferring during which substances are transferred out from the preacidifier and into the fermenter, whereby the sufficiently preacidified substances are selectively transferred.

The invention relates to an apparatus and a method for the anaerobic fermentation of materials.

An apparatus and a method according to the preamble of claim 1 and of claim 8 is known from the DE 198 04 007. This document relates to the treatment of an organically burdened fluid whereat the gas is generated in the fermentation stage. In practice it has turned out that the average duration of stay of the burdened fluids in the pre-acidification stage is approximately 15 hours and a total process approximately 30 hours. In this time the process water is sufficiently pre-acidified so that it can be transported into the fermenter without involving the risk that there would occur yet another acidification in the fermenter which would be very disadvantageous for the fermentation process particularly by damaging the methane producing bacteria due to low pH-values. Usually in the pre-acidification a pH-value of 6.0 or below must be reached. The pre-acidification serves to solubilize complex carbon compounds since the fermentation bacteria can utilize simple carbon compounds only.

During the fermentation biogas is extracted which represents a mixture of methane and carbon dioxide and which can be used for generating energy.

Further, from practice solid reactors are known in which pre-acidification and fermentation for the extraction of biogas take place jointly. In such reactors solid to pasty materials can be treated, whose time of stay is weeks until a sufficient total process has taken place.

At breweries fluid burdened process waters for example from cleaning processes for example and as more solid organic materials yeasts and draff, i.e. brewer grain for example arise. At fruit juice extraction plants also press residues like solid peel particles arise besides the process water. At such or similar production sites at which organically burdened fluids as well as organic remains arise normally both i.e. two independent reactors are therefore required primarily due to the incompatible pre-acidification periods in order to utilize the materials for biogas production. This is however quite costly and complicated.

The DE 199 37 876 A1 discloses a method for the biological conversion of organic materials to methane gas, which method is to achieve a power enhancement by shifting the stable operating point of the system. For that purpose the residence time of organic components in the reactors is to be uncoupled from the hydraulic residence time by the retention of organic components by means of a semipermeable membrane in order to achieve a better adaptation of the bacteria growth.

The object of the present invention is therefore to create an apparatus and a method with which a more cost effective simpler alternative for the production of biogas from fluid and solid materials can be realized.

This objective is achieved by an apparatus with the characteristics of claim 1 and a method with the characteristics of claim 8.

Preferred embodiments are disclosed in the respective sub claims.

With the apparatus according to the present invention means for transporting materials into the fermenter are provided with which it is possible by selective process control to transport all materials in a sufficiently pre-acidified condition. Here, still insufficiently pre-acidified normally more solid sparsely solubilized materials are left in the pre-acidifier for sufficient pre-acidification.

Accordingly the method according to the present invention relates to such one at which only sufficiently pre-acidified materials are transported.

In this way it is possible to put fluid respectively largely solubilized materials as well as solid or pasty less solubilized ones into a pre-acidifier and subsequently feed them sufficiently pre-acidified to a common fermenter.

For the selective transport a variety of material characteristics can be utilized which distinguish the sufficiently pre-acidified materials from the still insufficiently pre-acidified materials.

While for example sufficiently pre-acidified solid or pasty materials are soaked by the pre-acidification solubilization and dissolve in the fluid the insufficiently pre-acidified solids are normally coarse grained and sedimenting, respectively. Thus, a selection can be done by sieving and treating for example whereat the sieve for example has to be designed so that the still insufficiently pre-acidified solids get caught in the sieve and the fluid and soaked materials can pass through the sieve.

Furthermore, it has turned out that the still insufficiently pre-acidified solids in the pre-acidifier deposit themselves on the bottom of the same if no agitation or stirring takes place in the pre-acidifier. This means that upon switch-off of the agitation or of the stirring normally taking place in the pre-acidifier the insufficiently pre-acidified materials gather in the lower portion, whereas the sufficiently pre-acidified materials gather in the upper portion whereby a selection of sufficiently pre-acidified materials by a withdrawal of materials in the upper portion of the pre-acidifier is rendered possible by carrying out the forwarding into the fermenter only during the “rest phases”.

It has furthermore turned out that by a flotation for example by blowing air or gas into the pre-acidifier from the bottom the solids preferably bloat on the surface so that also here a separation of still insufficiently pre-acidified materials from the sufficiently pre-acidified materials results so that a selection during transport by a withdrawal of materials from the lower portion of the pre-acidifier is possible.

A preferred embodiment with very coarse feedstock materials such as peels, grains or the like includes a pre-treatment preferably a mechanical pre-comminution of the solids with a chopper or a mill or the like. A pre-treatment particularly a mechanical pre-comminution facilitates the solubilization of the solid materials in the pre-acidifier.

Preferred embodiments of the apparatus according to the present invention and the method according to the present invention are explained on the basis of the accompanying figures, in which:

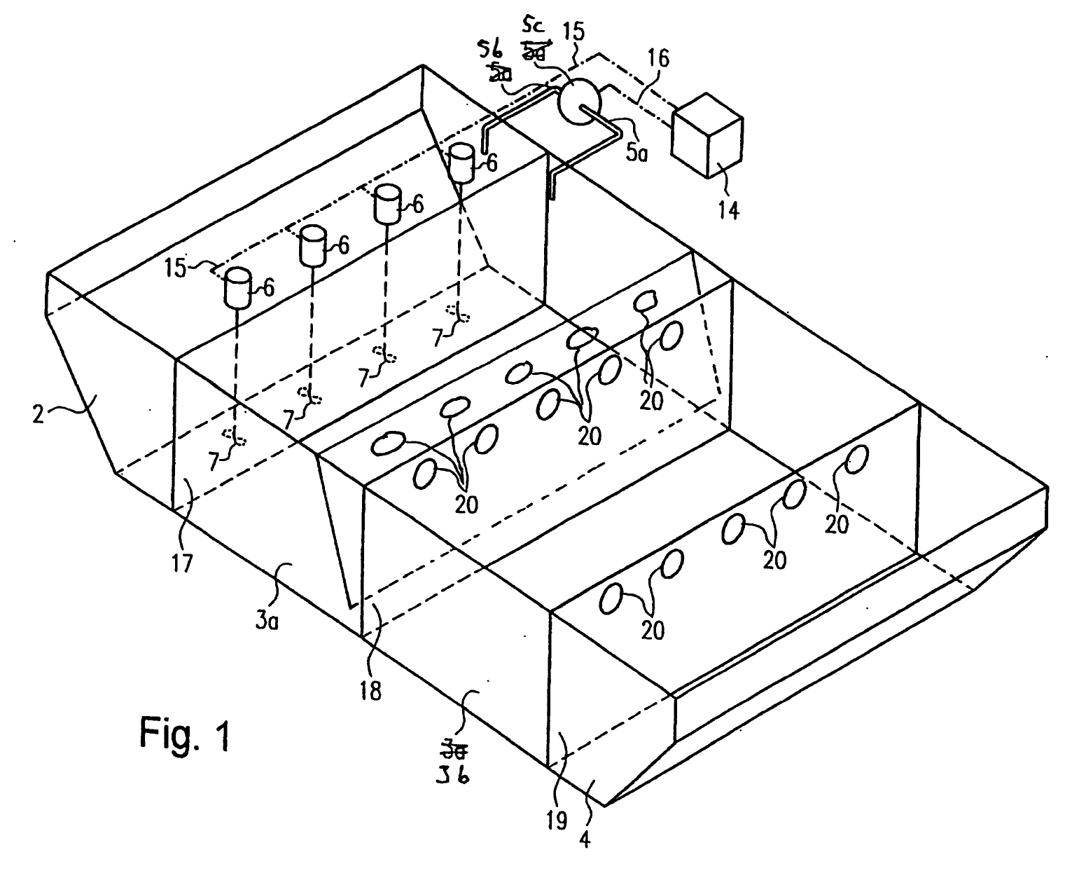

FIG. 1 shows a three-dimensional perspective view of an apparatus according to the invention,

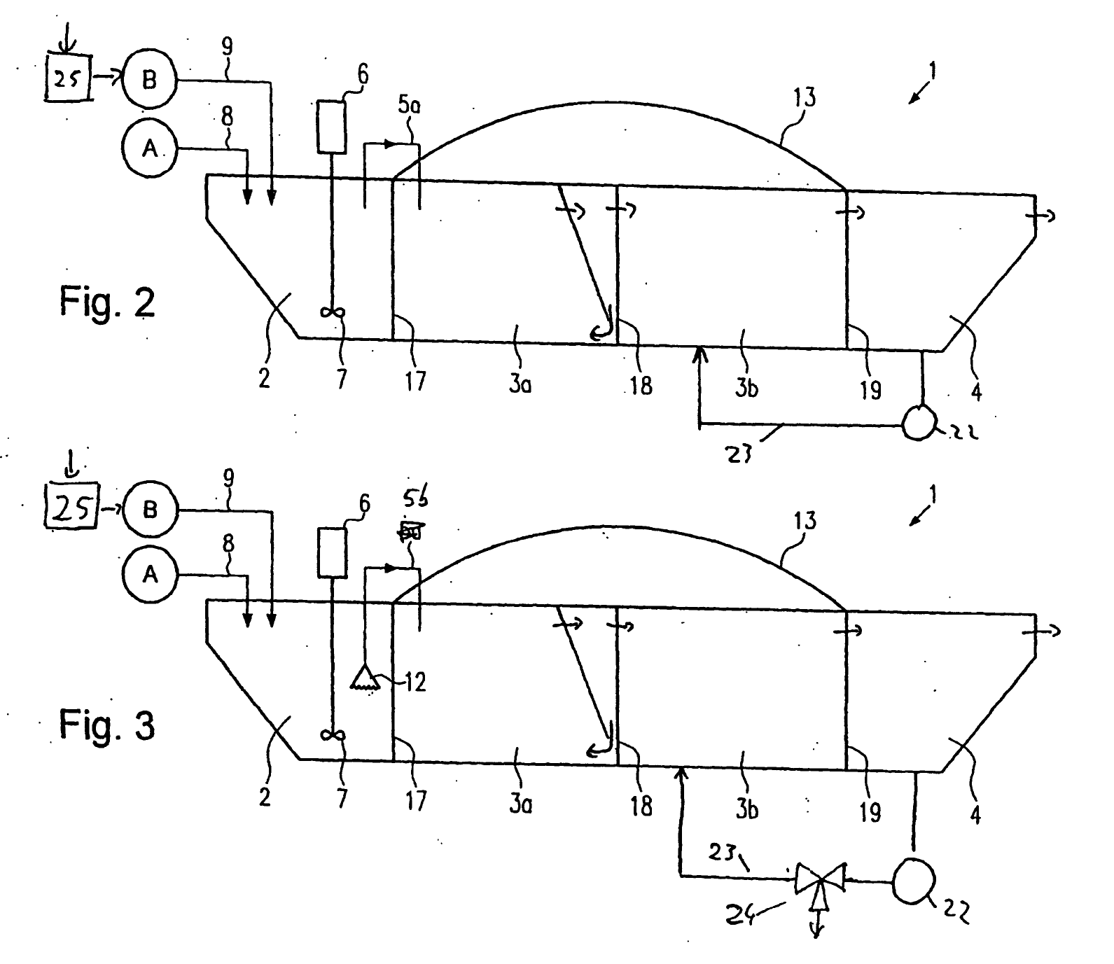

FIG. 2 shows a schematic sectional view of the apparatus according to the invention,

FIG. 3 shows a schematic sectional view of another embodiment according to the invention, and

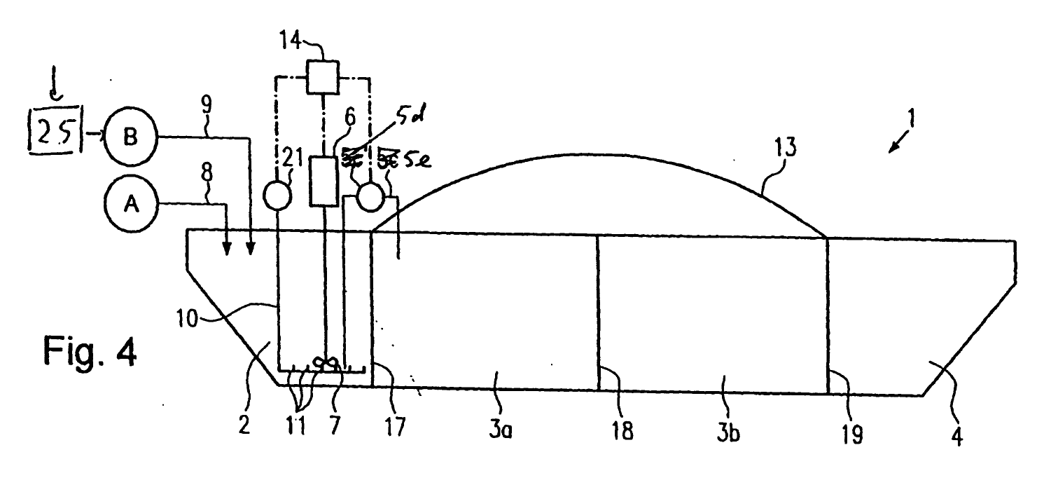

FIG. 4 shows a sectional view of a further embodiment of the invention.

In FIG. 1 an apparatus for the pre-acidification and anaerobic fermentation of materials is shown in a schematic three-dimensional illustration. The apparatus comprises a pre-acidifier 2, a fermenter with a main load stage 3a and a light load stage 3b and a final sedimentation stage 4. For the performance of the anaerobic fermentation main load stage 3a and light load stage 3b are covered by a tarpaulin for example for collecting and storing the arising gases, which is not illustrated in FIG. 1 for the sake of clarity. This tarpaulin can additionally cover pre-acidification 2 for example when the flotation occurs by means of gas or gas circulation (not illustrated).

Between pre-acidifier 2 and main load stage 3a a divider 17, between main load stage 3a and light load stage 3b a double divider 18 as well as between light load stage 3b and final sedimentation stage 4 a divider 19 are put in which can be adjustable or fixed.

In the portion of pre-acidifier 2 stirrers 6, 7 can be arranged with which an agitation can be carried out in the portion of pre-acidifier 2. Stirrers 6, 7 can be driven by a control device 14 via signal or power supply lines 15. Via a signal or power line 16 control device 14 can also be connected to a pump 5c with which a transport from pre-acidifier 2 into main load stage 3a via pipelines 5a and 5b can take place. At this the end of a nozzle and of a pipeline 5b are arranged in the portion of pre-acidifier 2 in the upper portion of pre-acidifier 2 i.e. for example above the middle along the height of pre-acidifier 2. Main load stage 3a as well as light load stage 3b can comprise partition walls not further illustrated. Double divider 18 between main load stage 3a and light load stage 3b as well as divider 19 between light load stage 3b and final sedimentation stage 4 may comprise transfer ports 20 or overflow spillways with which materials from the respective stage can pass over into the subsequent stage.

The basin illustrated in FIG. 1 can be embedded into the ground which however is not illustrated in figure 1 for reasons of clarity.

While in FIG. 1 pre-acidifier 2, fermenter 3a, 3b as well as final sedimentation stage 4 are accommodated in a basin they can also be arranged individually in a basin or tank.

In FIG. 2 there is a schematic sectional view of the apparatus of FIG. 1, in which tarpaulin 13 is illustrated which covers the main load stage and the light load stage. Under tarpaulin 13 the biogas produced can gather so that the tarpaulin bulges as illustrated in FIG. 2.

Furthermore in FIG. 2 the induction of the materials A and B via feed mechanisms 8 and 9 and for B alternatively via a pre-treatment 25, respectively, is schematically illustrated. While the fluid material A is normally fed through a pipeline the solid material B can be fed via a conveyor belt or via containers or the like. If the solid material B is available in a pasty or bloated form it can also be fed via a pipeline 9. In FIG. 2 a possible pre-treatment 25 of the solid materials B is also specified.

In FIG. 3 another embodiment of apparatus 1 is schematically illustrated. Here a sieve 12 is arranged at the entry of a pipeline 5b which serves for transporting from pre-acidifier 2 into main load stage 3a. While sieve 12 is arranged here in an approximately middle position with respect to the vertical height of pre-acidifier 2, it can also be arranged further up or further down. Sieve 12 is required to have a mesh aperture or transfer port opening size such that solids B which are fed into the pre-acidifier cannot pass through as long as they are insufficiently pre-acidified.

Instead of a pipeline 5b and a sieve 12 as transport means a sieve or a rack in a spillway or just a spillway with an automatically lockable valve can also be arranged through which materials from pre-acidifier 2 flow over into main load stage 3a. Such a spillway can be arranged at the upper end of divider 17 for example and can probably be lockable. The described spillways can also be arranged in the middle or lower part of divider 17.

Instead of a sieve 12 any other suitable device for separating coarse structured materials from soft or fluid materials as for example an intermediate sedimentation stage can also be provided. From such an intermediate sedimentation stage the solid materials would be transported back into pre-acidifier 2 so that they undergo a further pre-acidification.

FIG. 4 shows a further embodiment of the invention. At this a pipe 5e is provided as transport means, whose entry nozzle end is located in the lower portion of pre-acidifier 2. Furthermore a flotation device 10, 11 is provided with which air or gas which is fed through a pipeline 10 can be induced in the bottom portion of preacidifier 2.

A pump 5d is connected via a signal, a compressed-air, a hydraulic or a power line to a control device 14 which can drive pump 5d or an automatic valve (not illustrated). Furthermore, control device 14 is connected to an optional stirrer 6, 7 and/or to flotation device 10, 11, 21 via one or more signal, compressed-air, hydraulic or power lines and can drive these.

A first embodiment of a method according to the invention shall be explained on the basis of FIGS. 1 and 2.

Via pipelines 8 a brewery waste water (process water) for example is fed into the portion of pre-acidifier 2. The draff, yeasts and filtered out remains furthermore arising at the brewery can be fed into the portion of pre-acidifier 2 unprocessed, completely or partially pretreated via suitable transport means such as pipelines or conveyor belts or the like as solids B.

In this portion a fermentation by acidogeneous bacteria into mainly organic acids, hydrogen carbon dioxide as well as low alcohols occurs. At this a pH-value of approximately 6 and below or 5.5 and below is achieved.

To support the thorough mixing one or more stirrers 6, 7 for example are employed in the portion of pre-acidification 2.

The materials A and B are put into the portion of pre-acidifier 2 continuously or intermittently.

From pre-acidifier 2 a transport of materials into main load stage 3a must occur continuously or in intervals. For this, stirrers 6 and 7 or other agitation devices for example are switched off by means of control device 14. After a while (some minutes up to several ten minutes) the solids which are normally still insufficiently pre-acidified deposit themselves in the lower portion of pre-acidifier 2. In the upper portion of the reactor the sufficiently pre-acidified fluid materials as well as the soaked and therefore sufficiently preacidified solids gather. After the deposition in the portion of pre-acidifier 2 which is caused by a switch-off for example of stirrers 6, 7 a pump 5c is switched on via control device 14 which transports materials via pipelines 5a and 5b from the upper portion of pre-acidifier 2 into main load stage 3a or control device 14 opens the alternatively installed valve.

In main load stage 3a and in light load stage 3b the pre-acidified materials ferment and methanate, respectively, under the formation of biogas and the materials leaving light load stage 3b are final-treated in final sedimentation stage 4. The final-treated materials can be re-transported to the process, for example into the light load stage 3b, by means of pump 22 and pipeline 23 or taken out in a controlled way as surplus sludge by means of valve 24.

The draff and the filtered-out remains can be mechanically pre-hackled or purified otherwise so that for example a preset grain size of solid material results. This is advantageous to the controlled process control as well as to the accelerated pre-acidification of the solids.

On the basis of FIG. 3 a further embodiment of the method shall be explained. The addition of materials A, B to pre-acidifier 2 as well as the fermentation and final sedimentation is the same as the procedure described with reference to FIGS. 1 and 2.

In this method materials are transported continuously or in intervals from pre-acidifier 2 into main load stage 3a with a pipeline 5b and a pump, not illustrated, through a sieve 12. Sieve 12 retains the still insufficiently pre-acidified solids and lets the sufficiently pre-acidified fluid and soaked materials pass through.

At this the withdrawal from pre-acidifier 2 can be carried out in a middle position, as illustrated in figure 3, or further up or further down.

Furtheron, means for keeping sieve 12 free such as slides can be provided which remove in suitable intervals material accumulating on sieve 12. For that purpose a device can also be provided which creates a cross current in sieve 12 in order to remove accumulating material from sieve 12 in this way.

The agitation device, here stirrers 6, 7 may be switched on or switched off during the transport.

Using FIG. 4 a further embodiment of a method shall be explained.

Here the feed of the materials A, B and the fermentation and final sedimentation, respectively, are the same as described with reference to FIGS. 1 and 2.

For the transport of materials from pre-acidifier 2 into main load stage 3a a pump 21 for example with which air or gas is pumped through a pipeline 10 into the lower portion of the pre-acidifier is driven by control device 14. There the air or the gas exits through outlets 11 so that a flotation in pre-acidifier 2 occurs. At this juncture the insufficiently pre-acidified solids are bloated upwards. Meanwhile with control device 14 a pump 5d is put in operation which transports materials via pipelines 5e from pre-acidifier 2 into main load stage 3a. Here the entry of the nozzle to pump 5d lies in the lower portion of pre-acidifier 2, because here no insufficiently preacidified solids are present during the flotation. The nozzle can however also lie in the middle or upper portion when floating is not performed but sedimenting.

An optionally provided stirrer 6, 7 is preferably switched off during transporting.

With the method described above it is possible for example to set the mean duration of stay of fluid materials A in the pre-acidification 2 to approximately 5 to 15, advantageously approximately 10 hours, and to set the mean duration of stay of solids to between 30 and 150, preferably approximately 100 hours. Thereby it is possible to transport only sufficiently pre-acidified materials into main load stage 3a and simultaneously however jointly or individually process fluid as well as solid materials A, B.

It has turned out that with the above method a utilization of the solids for the production of biogas of up to 80% and more is possible.

The advantages in economical respects and in procedural/operational respects by reduction to one reactor line in the handling of inherently diverse organic materials are great.

1-13. (canceled)

14. Apparatus for the anaerobic fermentation of materials with a preacidifier in which said materials are subject to a preacidification, a fermenter in which said preacidified materials ferment, and transport means for transporting said materials from said preacidifier into said fermenter, wherein said transport means are formed to selectively transport sufficiently preacidified materials.

15. Apparatus according to claim 15, wherein said transport means comprise a withdrawal device for the withdrawal from the upper portion of said preacidifier, which are preferably formed by a spillway of said preacidifier or by a withdrawal nozzle end arranged in the upper portion of said preacidifier.

16. Apparatus according to claim 15, wherein said transport means comprise a control device for said withdrawal device, with which said withdrawal device and preferably an agitation device such as a stirrer can be driven.

17. Apparatus according to claim 14, wherein said transport means comprise a sieve.

18. Apparatus according to claim 14, wherein said transport means comprise a floatation device and a withdrawal device in the lower portion of said preacidifier.

19. Apparatus according to claim 18, wherein said transport means comprise a control device for said withdrawal device with which said withdrawal device and preferably said floatation device can be driven.

20. Apparatus according to claim 14, wherein a mechanical pretreatment, prehackling device for solubilizing/hackling at least part of said materials is provided.

21. Method for the anaerobic fermentation of materials with a preacidification at which said materials are preacidified with a preacidifier, a fermentation at which said pre-acidified materials ferment in a fermenter, and a transport at which materials from said preacidifier are transported into said fermenter, wherein said sufficiently preacidified materials are selectively transported.

22. Method according to claim 21, wherein the transport comprises letting said materials deposit themselves in said preacidifier and a subsequent withdrawal of materials from an upper portion of said preacidifier.

23. Method according to claim 21, wherein said materials are guided through a sieve during the transport.

24. Method according to claim 21, wherein the transport comprises a floatation and an at least partially simultaneous transport from the lower portion of said preacidifier.

25. Method according to claim 21, wherein said materials comprise fluids and solids.

26. Method according to claim 21, wherein at least part of said materials, particularly said solids are pretreated preferably mechanically prehackled before they are put into said preacidifier.
